(19) United States
(12) Patent Application Publication
(10) Pub. No.: US 2024/0341623 A1
(43) Pub. Date: Oct. 17, 2024

(54) SYSTEM AND METHOD FOR CONGESTIVE HEART FAILURE BIOMARKER DETECTION

(71) Applicant: Viking North Ventures, LLC, Dripping Springs, TX (US)

(72) Inventor: Joel D. Becker, Dripping Springs, TX (US)

(21) Appl. No.: 18/634,858

(22) Filed: Apr. 12, 2024

Related U.S. Application Data
(60) Provisional application No. 63/458,905, filed on Apr. 12, 2023.

(51) Int. Cl. A61B 5/08; A61B 5/00; A61B 5/097

(52) U.S. Cl. CPC A61B 5/082; A61B 5/097; A61B 5/743

(57) ABSTRACT

The present disclosure relates to a congestive heart failure biomarker detector. The congestive heart failure biomarker detector includes a gas sampling unit adapted to collect a gas sample for evaluating congestive heart failure. The congestive heart failure biomarker detector includes a chemical isolation unit adapted to selectively isolate a biomarker of interest from the gas sample to a predetermined level of accuracy. The congestive heart failure biomarker detector includes a processing unit configured to determine a level of the isolated biomarker of interest. The congestive heart failure biomarker detector includes a display unit configure to display the determined level of the isolated biomarker of interest.

### CROSS-REFERENCE TO RELATED APPLICATION

This application is based upon and claims priority to provisional application No. 63/458,905 filed on 12 Apr. 2023, the entire content thereof is incorporated herein by reference in its entirety.

### FIELD OF THE INVENTION

The present invention is related to the field of biomarker detection. In particular, this invention relates to congestive heart failure biomarker detection.

### BACKGROUND OF THE INVENTION

Heart failure is a chronic, progressive condition where the heart muscle is unable to pump enough blood to meet the body's needs for blood and oxygen leading to decreasing quality of life and possibly death. The incidence and prevalence of heart failure are both increasing due to underlying disease growth rates and demographics. Each time a person suffers from an episode of acute heart failure and needs to be hospitalized, their heart is further damaged, which may contribute to their heart failure getting worse. Repeat hospitalizations for heart failure remain a strong predictor of illness and death for heart failure patients. Currently, more than six million adults in the United States have been diagnosed with heart failure. It is currently estimated to be the number one cause for hospitalization for patients over 65 in the United States.

Biomarker monitoring (Acetone) has been shown in clinical studies to be highly statistically significantly correlated to the current gold standards of monitoring. An understanding of techniques for collecting acetone samples from a human can be gleaned from U.S. Pat. No. 8,747,325 B2 issued to Bacal et al. on 10 Jun. 2014, hereby incorporated by reference in its entirety as though fully set forth herein. An understanding of breath biosensing can be gleaned from Gaffney, Erin M, Koun Lim, and Shelley D Minteer. “Breath Biosensing: Using Electrochemical Enzymatic Sensors for Detection of Biomarkers in Human Breath.” Current Opinion in Electrochemistry, Feb. 27, 2020, hereby incorporated by reference in its entirety as though fully set forth herein. An understanding of skim-emitted acetone detection can be gleaned from Y. Yamada, S. Hiyama, T. Toyooka, H. Onoe, and S. Takeuchi, “Skin-Emitted Acetone Detection Toward Self-Monitoring of Fat Metabolisms,” Research Laboratories, NTT DOCOMO, Inc., Japan and Institute of Industrial Science, The University of Tokyo, Japan, hereby incorporated by reference in its entirety as though fully set forth herein.

Despite the prevalence of heart failure, a noninvasive monitoring technology does not currently exist (e.g., like blood sugar monitoring for diabetes). Additionally, early monitoring and intervention in the management of heart failure has been shown to improve outcomes but today no outpatient monitoring is available. One example of an invasive device is the CardioMEMS™ device (CARDIOMEMS is a trademark owned by CardioMEMS, LLC). That device is used for monitoring the pulmonary artery pressure in patients with heart failure. However, the CardioMEMS™ device must be permanently implanted in a pulmonary artery.

Accordingly, there is a need for noninvasive monitoring technology using biomarker monitoring.

### SUMMARY

Examples of the present disclosure provides a system and method for congestive heart failure biomarker detection.

According to a first aspect of the present disclosure, a congestive heart failure biomarker detector is provided. The congestive heart failure biomarker detector may include a gas sampling unit adapted to collect a gas sample for evaluating congestive heart failure. The congestive heart failure biomarker detector may also include a chemical isolation unit adapted to selectively isolate a biomarker of interest from the gas sample to a predetermined level of accuracy. The congestive heart failure biomarker detector may further include a processing unit configured to determine a level of the isolated biomarker of interest. The congestive heart failure biomarker detector may additionally include a display unit configure to display the determined level of the isolated biomarker of interest.

According to a second aspect of the present disclosure, a method for monitoring acetone levels in a patient diagnosed with congestive heart failure using a portable skin gas analyzer is provided. The portable skin gas analyzer may collect a plurality of gas samples from an area of skin of the patient over a predetermined period of time. The portable skin gas analyzer may also selectively isolate acetone from each collected skin gas sample in the plurality of skin gas samples to a predetermined level of accuracy. The portable skin gas analyzer may further determine a level of the acetone isolated from each collected skin gas sample in the plurality of skin gas samples. The portable skin gas analyzer may additionally communicate the determined level of the acetone isolated from each collected skin gas sample in the plurality of skin gas samples to the patient and/or a patient caregiver.

According to a third aspect of the present disclosure, a method for monitoring acetone levels in a patient diagnosed with congestive heart failure using a portable breath analyzer is provided. The portable breath analyzer may collect a plurality of breath samples from the patient over a predetermined time period. The portable breath analyzer may also selectively isolate acetone from each collected breath sample in the plurality of breath samples to a predetermined level of accuracy. The portable breath analyzer may further determine a level of acetone isolated from each collected breath sample in the plurality of breath samples. The portable breath analyzer may additionally communicate the determined level of the acetone isolated from each collected breath sample in the plurality of breath samples to the patient and/or a patient caregiver.

These and other aspects and advantages will become apparent to those of ordinary skill in the art by reading the following detailed description, with reference where appropriate to the accompanying drawings. Further, it should be understood that the foregoing summary is merely illustrative and is not intended to limit in any manner the scope or range of equivalents to which the appended claims are lawfully entitled.

### BRIEF DESCRIPTION OF THE DRAWINGS

The accompanying drawings, which are incorporated in and constitute a part of this specification, illustrate examples consistent with the present disclosure and, together with the description, serve to explain the principles of the disclosure.

FIG. 1 is a block diagram of a congestive heart failure biomarker detector, according to an example of the present disclosure.

FIG. 2 is a flow chart illustrating a method for monitoring acetone levels in a patient diagnosed with congestive heart failure using a portable breath analyzer, according to an example of the present disclosure.

FIG. 3 is a flow chart illustrating a method for monitoring acetone levels in a patient diagnosed with congestive heart failure using a portable skin gas analyzer, according to an example of the present disclosure.

### DETAILED DESCRIPTION

Reference will now be made in detail to example embodiments, examples of which are illustrated in the accompanying drawings. The following description refers to the accompanying drawings in which the same numbers in different drawings represent the same or similar elements unless otherwise represented. The implementations set forth in the following description of example embodiments do not represent all implementations consistent with the disclosure. Instead, they are merely examples of apparatuses and methods consistent with aspects related to the disclosure as recited in the appended claims.

The terminology used in the present disclosure is for the purpose of describing particular embodiments only and is not intended to limit the present disclosure. As used in the present disclosure and the appended claims, the singular forms “a,” “an,” and “the” are intended to include the plural forms as well, unless the context clearly indicates otherwise. It shall also be understood that the term “and/or” used herein is intended to signify and include any or all possible combinations of one or more of the associated listed items.

The present disclosure relates to congestive heart failure (CHF) biomarker detection. In one or more embodiments of the present disclosure, biomarkers, such as acetone, are used to evaluate congestive heart failure through a person's acetone levels in their breath or skin. Biomarkers are biological molecules found in blood, other body fluids, or tissues that are used as a sign of a normal or abnormal process, or of a condition or disease. Biomarkers were first noted by early Greek physicians and further described by John Rollo, an English physician, in 1798 as an odor of decaying apple in the breath of a diabetic patient. Currently biomarkers are utilized and researched in the areas of nutrition, exercise, and some additional emerging therapies. In one or more embodiments, biomarker detection can include mobile, patient friendly, noninvasive technologies that can be used to track the status of heart failure via a patient's breath and/or skin. The present disclosure can enable more frequent monitoring and earlier stage management of heart failure patients to avoid cardiac decompensation, impact heart failure disease progression, reduce rehospitalization rates, reduce costs, provide an improved quality of life for the patient, and reduce mortality.

Early detection of worsening heart failure enables more proactive treatment and reduces the risk of rehospitalization. Previously, physiologic indicators could only be measured in the hospital with a catheter placed above the heart and connected to a machine at the bedside, through a device implanted in the pulmonary arteries or through laboratory based blood analysis. Biomarkers (e.g., EBA, BNP) have been shown to be correlated with pulmonary capillary wedge pressure (PCWP) and worsening prognosis. In an embodiment, the EBA monitoring technology disclosed is dedicated to providing outpatient, mobile heart failure monitoring and reporting.

Biomarker monitoring, including acetone, has been shown in clinical studies to be highly statistically significantly correlated to the current gold standards of monitoring. Ketone metabolism is a biological process that occurs in the liver, where fatty acids are broken down into ketone bodies—mainly acetoacetate, beta-hydroxybutyrate, and acetone. These ketone bodies are then released into the bloodstream and transported to various tissues, including the brain, heart, and muscles. Acetone, produced during ketosis, can be used to monitor congestive heart failure. In individuals with CHF, the heart's ability to pump blood effectively is compromised, leading to a cascade of metabolic and physiological changes. One such change can be an altered energy metabolism, including the increased metabolism of fatty acids into ketones, of which acetone is a byproduct.

Biomarkers, such as acetone, can be used for heart failure therapy devices and methods of monitoring heart failure patients. In one or more embodiments, the use of biomarkers can include highly sensitive/accurate acetone monitoring equipment. Such equipment may allow for the design of a variety of heart failure treatment regimens and the systems for carrying out treatment regimens. The present disclosure includes different modalities that may be used to collect the acetone information at the core of the disclosed invention.

The congestive heart failure biomarker detector is intended to monitor, detect, analyze, and communicate the levels of exhaled breath acetone (EBA) present in the respiration of patients experiencing acute or chronic heart failure. This breath detected acetone levels are caused by the failing heart's increased energy requirements for which it metabolizes hepatic ketones to generate that energy and then take the form of acetone which is expired though the lungs of the heart failure patient. The congestive heart failure biomarker detector may include a gas sampling unit, a filtering and chemical isolation part, an analysis unit, a processing unit, a display unit, and a communications unit.

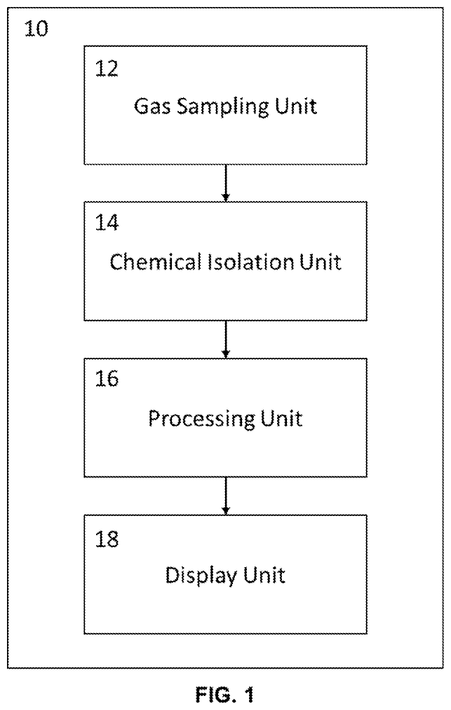

Turning now to the figures, FIG. 1 shows a congestive heart failure biomarker detector 10 according to an embodiment of the present disclosure. The congestive heart failure biomarker detector 10 includes a gas sampling unit 12, a chemical isolation unit 14, a processing unit 16, and a display unit 18. The gas sampling unit 12, for example, is a chamber for receiving a patients breath when a patient blows into the biomarker detector 10 (or breathalyzer type device). In such example, the gas sampling unit 12 may filter out moisture and other chemicals before analyzing for the biomarker. The gas sampling unit 12, in another example, may be part of gradient-based colorimetric array sensor (GCAS) (described below) which makes contact with a patients skin for detecting a biomarker. In an embodiment, the congestive heart failure biomarker detector gas sampling unit 12 collects a gas sample from a patient diagnosed with or experiencing the symptoms of heart failure. The gas sample can include a patient's breath sample. The chemical isolation unit 14 of the congestive heart failure biomarker detector can include a filter that filters out all non-essential organic compounds from the breath and isolates acetone selectively and to a level that allows the needed level of measurement accuracy of the compound of interest which may be acetone to a minimum detection level of 50 ppb. The congestive heart failure biomarker detector may also include an analyzing unit (not shown) comprised of a filtration component which may include Pt, Pd, or Au elements that are intended to isolate acetone and a sensor component which may be a semiconductive metal oxide (SMO) sensor which provides a detection signal corresponding to a level of chemical concentration of a heart failure biomarker which may be the level of acetone present in the collected breath sample of the congestive heart failure patient, receives the signal and evaluates it for breath sample volume, humidity, and the presence of other potential chemicals that could pollute the breath sample. The processing unit 16 of the congestive heart failure biomarker detector may provide information as to the level of breath acetone as a biomarker and track data from test results regarding the trend of acetone concentrations in breath samples due to the production of acetone to supply the energy needs of the failing heart. The display unit 18 shows the level of the measured and analyzed biomarker of interest (which may be acetone) to both the patient and clinician along with an indication of whether the measured value has been increasing or decreasing on a daily and/or moving average basis and may provide trending information as to the potential stage and progression of the patient's heart failure. The congestive heart failure biomarker detector 10 can further include a communications unit (not shown) that is able to remotely and wirelessly communicate the measured acetone readings to a computing device for notifying or communicating to both the patient and designated clinician the detected levels. The computing device may be a mobile device, personal computer, or server. The congestive heart failure biomarker detector system is designed to be incorporated into the patient's phone to a secure and HIPAA compliant software application that will also send the information to the patient's clinician with the potential to be integrated with the providers electronic health records and reimbursement system. The communications unit of the congestive heart failure biomarker detector may also contain notifications or alarms with specific notifications to the patient and/or provider if the measured acetone exceed preset limits as well as prompts to remind the patient to take their breath sample daily and the clinician to review the information when received.

### Exhaled Breath Acetone (EBA)

In one or more embodiments, biomarker detection can be done through a breath monitoring system. EBA can be used as a noninvasive biomarker that can reflect the metabolic state of an individual. Elevated levels of breath acetone have been associated with increased oxidative stress and a shift towards fatty acid metabolism, both of which are characteristics of the altered metabolic state in heart failure. Monitoring breath acetone levels can thus provide insights into the metabolic status of patients with CHF, potentially offering a convenient, noninvasive method to assess disease progression or response to treatment. Breath monitoring can be used to detect more than 300 volatile organic compounds. Clinical research has shown EBA levels to be higher in heart failure patients. EBA levels have been shown to increase with the number of Heart Failure Symptoms and highly statistically correlated with the gold standards of b-type natriuretic peptide (BNP) and pulmonary capillary wedge pressure (PCWP). Increased levels of EBA have also been shown to be associated with a worse prognosis for the progression (p=0.001) morbidity and mortality (p=0.001) of heart failure. To date, most clinical breath monitoring has needed to take place in either a clinic/in-patient setting or with shipped samples.

EBA has been shown to be correlated with the gold standards of heart failure monitoring (BNP, PCWP) and increasing levels of EBA correlated to a worsened patient prognosis. Advances in the accuracy and sensitivity of various types of chemical sensors has enabled increases in the selectivity and accuracy of breath monitoring. Thus, in one or more embodiments, EBA can be used for patients diagnosed with or experiencing symptoms of heart failure. For example, highly sensitive EBA detection can be done through the breath of a patient blowing on a device. The results of the EBA analysis is transmitted via Bluetooth to a mobile device or computing system for further analysis or reporting. For example, the results can be displayed on a mobile device. The results can also be transmitted to a clinician's Electronic Medical Records (EMR) systems with a notification. The results may also be sent to a server for technical interpretation or further analysis. Additionally, the results may be integrated with reimbursement systems, for example, it may be integrated with an Electronic Health Record systems that may then further take the digital test results and send to them to payors (insurers, CMS) systems as submittals for reimbursement to the care provider.

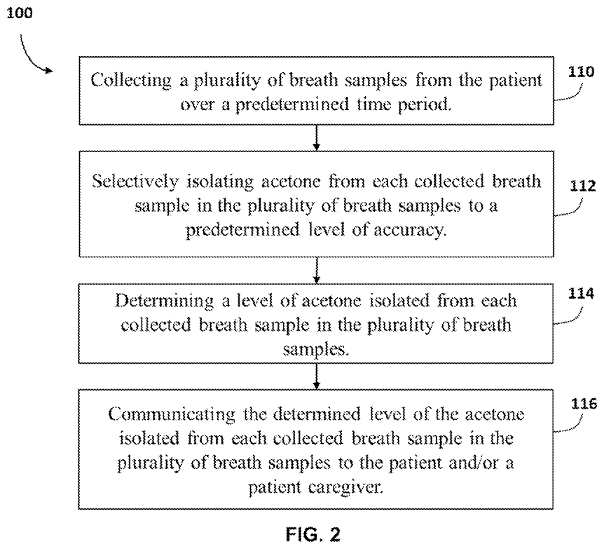

FIG. 2 shows an example method for monitoring acetone levels in a patient diagnosed with congestive heart failure using a portable breath analyzer in accordance with the present disclosure.

In step 110, the portable breath analyzer collects a plurality of breath samples from the patient over a predetermined time period. For example, the breath analyzer may be a device the patient places on their mouths and blows air into.

In step 112, the portable breath analyzer selectively isolates acetone from each collected breath sample in the plurality of breath samples to a predetermined level of accuracy. For example, the analyzer isolates the level of acetone for determining the level of concentration of acetone within a range of accuracy.

In step 114, the portable breath analyzer determines a level of acetone isolated from each collected breath sample in the plurality of breath samples.

In step 116, the portable breath analyzer communicates the determined level of the acetone isolated from each collected breath sample in the plurality of breath samples to the patient and/or a patient caregiver. The determined level is communicated, for example, by displaying a color indicator that is associated with a detected level. For example, the color indicator may be green if the levels are normal but red if they are not normal. In another example, the determined level is communicated by using a display on a computing device by displaying a numerical value or text related to the detected level.

### Skin Acetone Monitoring (SAM)

In one or more embodiments, biomarker detection can be done through a skin monitoring system. Skin monitoring has been shown to be able to detect transdermal acetone in patients with diabetes and undergoing ketosis. Monitored levels have been shown to be responsive to the treatment of the underlying condition.

In an embodiment, a gradient-based colorimetric array sensor (GCAS) can detect a biomarker such as a acetone for monitoring CHF. A GCAS is a type of chemical sensor that uses changes in color to detect and quantify the presence of various substances. These sensors incorporate a systematic variation, or gradient, in their sensing elements, which are often made of chemically responsive dyes. The transdermal acetone response from a GCAS correlates well with breath acetone in the range between 0 and 40 ppm. For example, breath acetone and skin acetone levels are well correlated within the range of 0 and 40 ppm thereby showing that a transdermal (wearable) approach would be viable to use with skin based sensing. A GCAS can be a noninvasive, low cost, wearable tool that could be applied for disease management. For example, the GCAS can be part of a type of wrist watch that the patient wears.

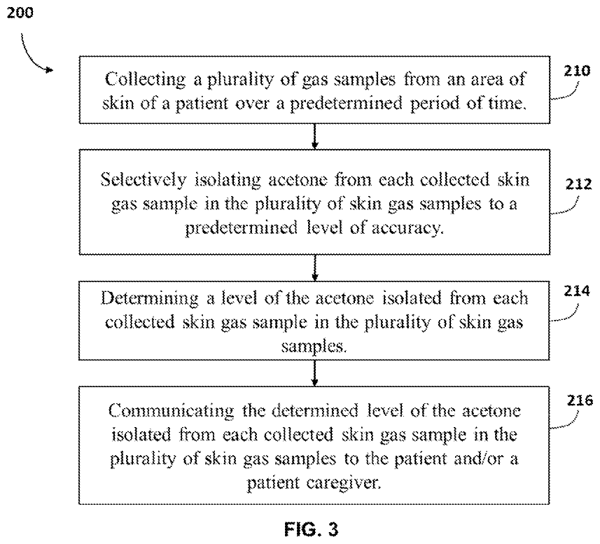

FIG. 3 shows an example method for monitoring acetone levels in a patient diagnosed with congestive heart failure using a portable skin gas analyzer in accordance with the present disclosure.

In step 210, the portable skin gas analyzer collects a plurality of gas samples from an area of skin of the patient over a predetermined period of time. For example, the portable skin analyzer may enclose an area of the patients skin to acquire the gas sample.

In step 212, the portable skin gas analyzer selectively isolates acetone from each collected skin gas sample in the plurality of skin gas samples to a predetermined level of accuracy.

In step 214, the portable skin gas analyzer determines a level of the acetone isolated from each collected skin gas sample in the plurality of skin gas samples.

In step 216, the portable skin gas analyzer communicates the determined level of the acetone isolated from each collected skin gas sample in the plurality of skin gas samples to the patient and/or a patient caregiver. The determined level is communicated, for example, by displaying a color indicator that is associated with a detected level. For example, the color indicator may be green if the levels are normal but red if they are not normal. In another example, the determined level is communicated by using a display on a computing device by displaying a numerical value or text related to the detected level.

What is claimed is:

1. A congestive heart failure biomarker detector comprising the following:
a gas sampling unit adapted to collect a gas sample for evaluating congestive heart failure;
a chemical isolation unit adapted to selectively isolate a biomarker of interest from the gas sample to a predetermined level of accuracy;
a processing unit configured to determine a level of the isolated biomarker of interest; and
a display unit configure to display the determined level of the isolated biomarker of interest.

2. The congestive heart failure biomarker detector of claim 1, further comprising:
a communications unit configure to communicate the determined level of the isolated biomarker of interest to a computing device.

3. The congestive heart failure biomarker detector of claim 1, wherein the gas sample comprises a breath gas sample from a patient diagnosed with or experiencing the symptoms of heart failure.

4. The congestive heart failure biomarker detector of claim 3, wherein the gas sampling unit comprises a portable breath analyzer.

5. The congestive heart failure biomarker detector of claim 1, wherein the gas sample comprises a gas sample emitted from an area of skin of a patient diagnosed with or experiencing the symptoms of heart failure.

6. The congestive heart failure biomarker detector of claim 5, wherein the gas sampling unit comprises a portable skin gas analyzer.

7. The congestive heart failure biomarker detector of claim 1, wherein the biomarker of interest is acetone.

8. The congestive heart failure biomarker detector of claim 7, wherein the predetermined level of accuracy is 50 parts per billion (ppb).

9. The congestive heart failure biomarker detector of claim 1, wherein the display unit comprises a color indicator for displaying the level of the isolated biomarker of interest.

10. The congestive heart failure biomarker detector of claim 1, wherein the display unit comprises a display configured to show trend information for the determined level of the isolated biomarker of interest, including showing whether the level has been increasing or decreasing on a daily and/or moving average basis.

11. A method for monitoring acetone levels in a patient diagnosed with congestive heart failure using a portable skin gas analyzer, the method comprising the following:
collecting a plurality of gas samples from an area of skin of the patient over a predetermined period of time;
selectively isolating acetone from each collected skin gas sample in the plurality of skin gas samples to a predetermined level of accuracy;
determining a level of the acetone isolated from each collected skin gas sample in the plurality of skin gas samples; and
communicating the determined level of the acetone isolated from each collected skin gas sample in the plurality of skin gas samples to the patient and/or a patient caregiver.

12. The method of claim 11, further comprising:
creating a care regime for the patient based at least in part on the determined levels of acetone isolated from each collected skin gas sample in the plurality of skin gas samples.

13. The method of claim 11, wherein the predetermined level of accuracy is 50 ppb.

14. The method of claim 11, wherein the communicating step comprises displaying the determined level of the acetone isolated from each collected skin gas sample in the plurality of skin gas samples.

15. A method for monitoring acetone levels in a patient diagnosed with congestive heart failure using a portable breath analyzer, comprising:
collecting a plurality of breath samples from the patient over a predetermined time period;
selectively isolating acetone from each collected breath sample in the plurality of breath samples to a predetermined level of accuracy;
determining a level of acetone isolated from each collected breath sample in the plurality of breath samples; and
communicating the determined level of the acetone isolated from each collected breath sample in the plurality of breath samples to the patient and/or a patient caregiver.

16. The method of claim 15, further comprising:
creating a care regime for the patient based at least in part on the determined levels of acetone isolated from each collected breath sample in the plurality of breath samples.

17. The method of claim 15, wherein the predetermined level of accuracy is 50 ppb.

18. The method of claim 15, wherein the communicating step comprises displaying the determined level of the acetone isolated from each collected breath sample in the plurality of breath samples.
